(19) United States
(12) Patent Application Publication
(10) Pub. No.: US 2021/0177031 A1
(43) Pub. Date: Jun. 17, 2021

(54) AN ARRAY OF AGE-TAILORED NUTRITIONAL FORMULA FOR INFANTS AND YOUNG CHILDREN FOR USE IN THE PREVENTION OF SUB-OPTIMAL BODY COMPOSITION

(71) Applicant: SOCIETE DES PRODUITS NESTLE S.A., Vevey (CH)

(72) Inventors: Jian Yan, Vevey (CH); Laura Gosoniu, Epalinges (CH); Philippe Steenhout, Leytron (CH)

(21) Appl. No.: 16/762,756

(22) PCT Filed: Nov. 7, 2018
(86) PCT No.: PCT/EP2018/080477

§ 371 Date: May 8, 2020

(30) Foreign Application Priority Data
Nov. 9, 2017 (EP) 17200838.5

(51) Int. Cl. A23L 33/00; A23L 33/12; A23L 33/19; A23L 33/135

(52) U.S. Cl. CPC A23L 33/40; A23L 33/12; A23L 33/135; A23L 33/30; A23L 33/19

(57) ABSTRACT

An array of nutritional compositions for infants and/or young children is proposed. Each composition of such array is targeted at a specific age of the infant/young child and it formulated such as to prevent sub-optimal body composition of the subject (especially in terms of fat mass and/or fat-free mass). By preventing sub-optimal body composition the array of the invention help preventing later in life undesired health conditions such as obesity, overweight, diabetes and diabetes related conditions, and/or cardiovascular diseases.

### FIELD OF INVENTION

The present invention relates to nutritional formulae which are specifically designed to address the needs of infants and young children. In particular, the invention provides an array of nutritional compositions for the infants and young children, each nutritional composition having an age-specific composition which varies according to the age of the infant/child.

The set of nutritional compositions is specifically aimed at providing long-term benefits to the infants and young children. Such benefits includes the prevention of sub-optimal body composition (especially in terms of fat mass and/or fat-free mass. This may be also linked to health benefits later in life such as reducing obesity, reducing cardiovascular diseases and reducing metabolic disorders associated with obesity later in life.

### BACKGROUND OF INVENTION

Mother's milk is recommended for all infants. However, in some cases breast feeding is inadequate or unsuccessful for medical reasons or the mother chooses not to breast feed. Infant formulas have been developed for these situations. The array of nutritional composition of the present invention have also been developed for these situations.

Conventional nutritional compositions for infants and young children usually fall into two categories: Starter formulas (aka starter Infant formula (IF)) for infants from the age of birth to 4 to 6 months and which provide complete nutrition for this age group and so-called follow-on formulas (FOF) for infants between the ages of four to six months and twelve months. Later, so-called “Growing-Up Milk” are designed for infants more than a year old, usually up to 3 years. FOFs and GUMs can be fed to the young children in combination with increasing amounts of other foods such as infant cereals and pureed fruits, vegetables and other foodstuffs as the process of weaning progresses. Many of these commercially available nutritional compositions are based on cows' milk proteins and contain whey and/or casein proteins although others are based on soy proteins. Where both whey and casein proteins are present, the ratio between them may vary between 90:10 and 10:90.

Infant formulae, follow-up formulae and grown-up milks which may be aimed at different age groups of 0 to 6 months, 6 months to 1 year and 1 year to 3 years respectively, are known. These infant formulae, follow-up formulae and grown-up milks aim to meet the requirements of infants and young children at the different ages.

An age-tailored nutrition system for infants is described in WO2009/068549, wherein a protein nature and content are adapted to specific age groups. The array of compositions of WO2009/068549 can be used in the context of the present invention.

The recommended World Health Organisation (WHO) and Codex Alimentarius Commission guidelines for infant formulae and follow-up formulae state the recommended basic values for nutrients such as protein content, fat content, carbohydrate content, energy density, as well as micronutrient (vitamins, minerals etc. . . . ). Similarly the European directives on infant formula and follow on formula provide guidance.

The relevant industry has developed and commercializes a vast array of nutritional compositions for infants and young children. Usually these compositions are proposed in a dry format (powder) that is intended to be reconstituted into water in specific proportions. Alternatively these compositions are provides in a liquid format, either ready-to-use or intended to be diluted into water.

There is however generally a tendency to overfeed infants and young children. Baby bottles are commonly used by care-givers as a way to pacify the infants and overfeeding is not uncommon.

Usually also, care givers may be temped to overpass the recommended dosage (for example in case of powder to be diluted. Many parents typically provide “one additional spoon of powder” into the baby bottle, with the best intention.

Usually also, the infant formulae and/or follow-up formulae dosage and caloric density do not take into consideration the complementary food eaten by infants and young children when overfeeding the infants and young children. Importantly also the best infant nutritional compositions for infant and young children are still not totally equal to human breast milk in term of nutritional value. Human breast Milk (HBM) is indeed highly complex and its secrets have not yet all been decrypted: In view of the immense diversity of the HBM constituents, some in trace amounts but nutritionally important, any synthetic nutritional formula currently available can only be considered as a nutritional approximation.

Finally the human breast milk evolves drastically over time (over the age of the breast fed infant). The changes over time of the multiple constituents of HBM are still to be fully understood and evidently only those parameters that have been scientifically measured have been highlighted as being important.

Synthetic infant nutritional compositions are usually Mother Nature and the human breast milk remains the gold standard.

If it is known that conventional synthetic infant formulas are able to induce a rapid growth of infants. It is also known that a rapid growth of infants and young children increases a risk of obesity in later childhood or adulthood (see Baird et al.; Being big or growing fast: systematic review of size and growth in infancy and later obesity. BMJ. 2005; 331(7522):929).

Similarly studies have shown that conventional infant nutritional formulation may induce a body composition (in terms of fat-mass and/or fat free mass) that is different from the body composition of breast fed infants.

Gale et al. conducted a systematic review and meta-analysis of more than 10 studies and highlighted the undesirable effect (see Gale et al.; effect of breastfeeding compared with formula feeding on infant body composition: a systematic review and meta-analysis. Am J Clin Nutr 2012; 95:656-69). Specifically, fat mass is reported to be lower in formula-fed (vs. breastfed) infants at age 3-4 mo (months) and 6 mo, and to be higher in formula-fed (vs. breastfed) infants at 12 mo. Fat-free mass is reported to be higher in formula-fed (vs. breastfed) infants at 3-4 mo, 8-9 mo, and 12 mo. Additionally, the percentage of fat mass is reported to be lower in formula-fed (vs. breastfed) infants at 3-4 mo and 6 mo.

Body composition at young age is an important parameter which is linked to various sub-optimal status later in life: over-weight obesity, cardiovascular diseases, metabolic syndrome, diabetes, insulin resistance and the like. Hence there is a need to provide (synthetic) nutritional compositions to infants and young children, which prevent sub-optimal body composition, especially in terms of fat-mass and/or fat-free mass.

There is a need to reduce the risk of over-weight, obesity, cardiovascular diseases, diabetes and related status, in later childhood or adulthood.

Experiments in animals have shown that modification in energy intake in the first weeks of life has a lifelong effect on weight gain even if normal energy intake was restored afterwards in later life (see Widdowson E M and McCance R A The effect of finite periods of under nutrition at different ages on the composition and subsequent development of the rat. Proc R Soc Lond B Biol Sci 1963 (1); 158:329-342)

A number of studies indicate that nutrition in early postnatal life has an impact on long-term appetite regulation. For example, overfeeding rat pups results in altered appetite control with development of hyperphagia in adulthood of the rats (see Davidowa H, Plagemann A; Hypothalamic neurons of postnatally overfed, overweight rats respond differentially to corticotropin-releasing hormones. Neurosci Lett. Nov. 16, 2004; 371(1):64-8).

There is a need to provide a more balanced diet to infants and young children. The diet should promote health benefits in the long term to the infants and young children.

There is a need to provide a nutritional system that enables the convenient, safe and accurate delivery of the most adequate nutrition all along the first months or years of the life of a baby.

There is a need for these systems to be easily complied with by the care givers, reducing the risk of over-feeding.

There is need to provide a way to insure that the best adequate individual nutritional solutions are made available to infants and their care-givers, in order to promote health benefits that may not be immediately visible but which consequences occur later in life.

There is a need to provide such cited nutritional compositions, especially during the first 6 months of life and/or during the first 12 months of life, that can help insuring best growth and reduction of risk of health conditions later in life, such as cardiovascular diseases, diabetes, obesity, or metabolic syndrome.

There is a need to provide a nutritional system that encompasses more than the first few months of life and acknowledge the introduction of complementary food (i.e. non infant formula) in the diet of the babies, while insuring best growth and reduction of risk of health conditions later in life, such as cardiovascular diseases, diabetes, obesity, metabolic syndrome, or depressed immunity.

There is a need for an early-in-life nutritional intervention or control in order to deliver health benefits later-in-life.

There is a need to avoid, prevent and/or reduce the risk of sub-optimal building of fat-mass (respectively promoting fat-free mass) among infants and young children.

There is a need to promote a fat mass and/or fat free mass which is comparable to infants (of similar genetic and/or ethnic origins) who are exclusively breast-fed, breast fed in a large proportion, or breast-fed during an extended period of time (6 months or more).

For the benefit of infants that will not be completely breast fed in the first few months of life, there is a continuing need to develop infant formulas which will replicate human milk as far as possible in terms of its nutritional properties, in particular such as to prevent an sub-optimal body composition (especially in terms of fat mass/fat-free mass).

An object of the present invention is to provide a nutritional solution for infants and young children that ensure the prevention of undesired health effects such as sub-optimal body composition.

It is also an aim of the present invention to provide long-term health benefits to infants and young children.

### SUMMARY OF INVENTION

The invention relates to an array of nutritional compositions for use, by infants/children in their first 2 to 3 years of life, in the prevention of sub-optimal body composition. The array can comprise
A first infant composition for use during the first month of life of the infant, and
A second infant composition for use during the second month of life of the infant, and
A third infant composition for use during third to sixth months of life of the infant, and
An optional fourth infant composition for use during 7-12 months of age of the infant/young child, and
An optional fifth and/or sixth composition for use respectively during the 13^{th} to 24^{th} months and 25th to 36^{th} months of life of the young child, and
wherein the protein and/or the fat content and/or the energy density varies between the compositions to reflect the evolving changes of breast milk over age of the infant/young child.

In a further aspect the invention relates to the long term prevention of sub-optimal states such as cardiovascular diseases, obesity, overweight, diabetes and related states.

In a further aspect the invention relates to inducing a fat mass and/or a fat-free mass in infants and young children under development that is the most appropriate, ie that is comparable, similar of identical to the fat-mass and/or fat-free mass of the infants and young children that are/were exclusively or essentially breast-fed during the first 4, 6, or 12 months of their life.

In a yet further aspect the invention related to the method of providing nutrition to infants and young children such as to prevent/avoid sub-optimal body composition (especially related to fat-mass and/or fat-free mass) later in life, by feeding them tan age tailored array of composition.

In a yet further aspect, the invention relates to kit and/or a system using the array of composition and delivering the cited prevention of undesired health conditions.

### BRIEF DESCRIPTION OF THE FIGURES

FIG. 1 shows the fat-mass percentage and fat-free mass percentage of infants at 3 months and 4 months—comparing infants having received the array of compositions of the invention to breast-fed infants.

FIG. 2 shows the serum markers levels between infants fed with the compositions of the invention and breast-fed infants.

### DETAILED DESCRIPTION OF THE INVENTION

### Definitions

As used herein, the following terms have the following meanings.

The term “infant” means a child under the age of 12 months.

The term “young child” means a child aged between one and three years.

The term “body composition” is to be understood as the composition of the body in terms of fat-mass and fat-free mass, unless otherwise provided.

The term “infant formula” means a foodstuff intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae).

The term “follow-on formula” means a foodstuff intended for particular nutritional use by infants aged over four months and constituting the principal liquid element in the progressively diversified diet of this category of person.

The term “starter infant formula” means a foodstuff intended for particular nutritional use by infants during the first four months of life.

Infant formula follow on formula and starter infant formula can either be in the form of a liquid, ready-to-consumer or concentrated, or in the form of a dry powder that may be reconstituted to form a formula upon addition of water. Such formulae are well-known in the art.

The term “baby food” means a foodstuff intended for particular nutritional use by infants during the first years of life.

The term “infant cereal composition” means a foodstuff intended for particular nutritional use by infants during the first years of life.

The term “growing-up milk” means a milk-based beverage adapted for the specific nutritional needs of young children.

The term “weaning period” means the period during which the mother's milk is substituted by other food in the diet of an infant.

The term “nutritional composition” means a composition which nourishes a subject. This nutritional composition is usually to be taken orally or intravenously, and it usually includes a lipid or fat source and a protein source. Preferably the nutritional composition is a complete nutrition mix that fulfils all or most of the nutritional needs of a subject (for example an infant formula).

The terms “nutritional composition”, “infant formula”, “follow-on formula”, growing up milk” and the like, are to be understood as “man-made”, i.e. synthetic nutritional compositions, and do not encompass human breast milk.

The term “synthetic mixture” or “synthetic composition” means man-made mixtures obtained by chemical and/or biological means, which can be chemically identical or similar to the mixture naturally occurring in mammalian milks.

The term “oligofructose” (abbreviated OF) as used herein refers to a fructose oligomer (i.e. a fructose oligosaccharide) having a degree of polymerization of from 2 to 10, for example a degree of polymerization of from 2 to 8. Oligofructose can also be referred as Fructo-Oligo-Saccharides (abbreviated FOS) or short-chain Fructo-Oligo-Saccharides (abbreviated scFOS). In the present document the terms oligofructose (OF), fructose oligosaccharide (FOS), Fructo-Oligo-saccharide (FOS), short-chain-fructo-oligosaccharide (scFOS) have the same meaning and can be used interchangeably.

The Inulin, being polymers of long chains are specifically excluded from the present definition of OF. Oligofructose is distinguishable from Inulin by its degree of polymerization (Inulin having much longer chains).

FOS/scFOS/Oligofructose is typically commercially available, for example under the commercial name ORAFTI Oligofructose by Beneo GmbH (Mannheim, Germany) (for example ingredient Orafti® P95).

The term “sn-2 palmitate” as used herein refers to palmitic acid in the sn-2 position of the triglyceride to which it is bonded.

The term “sialylated oligosaccharide” means an oligosaccharide having a sialic acid residue.

The term “fucosylated oligosaccharide” means an oligosaccharide having a fucose residue.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10).

The term “array” of compositions is to be considered equivalent to the term “set” of compositions. It describes a physical or theoretical assembly/juxtaposition of different nutritional compositions intended to be used in combination with each other (especially at different age of the infants/young children). Its usage can be performed by the care giver (parents, nurses, health care professionals (HCPs) . . . ) in the context of home use, out-of-home use or in the infant care industry (nurseries, childcare centers, kindergartens, hospitals, etc. . . . ). The assembly/juxtaposition of the compositions can take the form of “physical sets” of compositions sold for example in one unique packaging, or sold separately but intended to be used in a sequential order by the same infants/young children at the specified age (and thus interacting synergistically to deliver the health benefits when used). It is to be noted that a professional care giver can use the various compositions of the array at the same time on different infants—each infant receiving the compositions targeted to its specify age group according to the invention.

The term “kit” refers to the “physical sets of compositions”. The compositions can be packed in individual single use capsules delivering one serving. The system commercially available at the time of the invention in PR China, France and Switzerland under the name BabyNes® by Nestle is an example of the use of such kits (see www.babynes.com).

The term “later in life” indicates that the health effect is delayed in comparison to the time of the nutritional intervention. Typically the health effect is measurable 4, 6, or 12 months, or 2, 3, 4 5, 7 or 10 years after the start of the nutritional intervention. In the context of the invention the body mass composition can be visible/measurable a few weeks (1, 2, 4, 6 or 8 weeks) or a few months (1, 2, 3, 4, 6, or 12 months) after the nutritional intervention. Typically the prevention of the undesired health effect (linked to the establishment of the most adequate body composition) is measurable 1, 2, 3, 5, 7 or 10 years after the nutritional intervention.

All percentages are by weight unless otherwise stated.

When the ingredients amounts are provided for as weight of ingredient/weight of powder nutritional composition is also intended that the invention comprises also the corresponding amount by litre taking in to account a dilution factor of the dry powder nutritional composition of 130 g/L (or a specified otherwise in the dilution instructions).

### Invention

For a complete understanding of the present invention and the advantages thereof, reference is made to the following detailed description of the invention. It should be appreciated that various embodiments of the present invention can be combined with other embodiments of the invention and are merely illustrative of the specific ways to make and use the invention and do not limit the scope of the invention when taken into consideration with the claims and the following detailed description.

### Form of the Compositions

The nutritional compositions are preferably in the form of a powder to be reconstituted or the nutritional compositions are in the form of a concentrate to be diluted. The powder or concentrate can be reconstituted or diluted with water. The end product is thus preferably a liquid. In an embodiment the nutritional compositions of the invention are liquid and ready-to-consumer, or liquid and to be diluted with water.

Typically, the compositions forming part of the array of nutritional compositions comprise any of a source of fat, proteins and/or carbohydrates or any mixtures thereof. The compositions usually further comprise vitamins and minerals. The compositions of the invention follow the usual guidelines (CODEX, European directives on infant formula etc. . . . ).

### Number of Compositions in the Array of Compositions of the Invention

Typically the array of the invention comprises at least 3 nutritional compositions, each targeted at a different age of the infants/young children, and each being different from the other compositions of the array by the nature and/or amount of at least one ingredient.

In various embodiments of the invention the array of compositions comprises 4 or 5 or 6 compositions. An array with 4 or 5 or 6 compositions may be even more suitable to induce the health effects of the invention as the duration of nutritional intervention is higher.

### Age Tailoring

Each composition of the invention is tailored to correspond and fulfils the nutritional needs of infants/young children at the specific age. Additionally each composition is intended to fulfil the generally recommended and regulatory requirements for the nutrition of infants7 young children of specific age.

The skilled person will understands that each composition is designed for a specific age and that providing the infants/young children with a composition that is not designed for his/her age is suboptimal and would not provide the best and complete nutrients necessary for the normal development of the infant/young child and would not avoid the undesirable health effects targeted by the present invention.

The first composition of the invention is intended for age 0 to 1 month of age, i.e. for the first month of life

The second composition of the invention is intended for age 1 to 2 months of age, i.e. for the second month of life.

The third composition of the invention is intended for age 2 to 6 months of age, i.e. for the third to sixth months of age.

The optional fourth composition of the invention is intended for age 6 to 12 months of age, i.e. for the 7^{th} to 12^{th} months of life.

The optional fifth composition of the invention is intended for age 12 to 24 months of age, i.e. for the 13^{th} to 24^{th} months of life.

The optional sixth composition of the invention is intended for age 24 to 36 months of age, i.e. for the 25^{th} to 36^{th} months of life.

### Energy Density

Energy density is expressed in kcal/100 ml of “ready to consume” composition (reconstituted as needed). The term “once reconstituted” refers to the ready to consumer compositions which have been reconstituted if necessary (for example in case of powder compositions, and for example with water). In case of nutritional compositions which are already “ready to consumer” (for example already diluted liquid compositions) the term “once reconstituted” means “ready to consume” (even if there is no need for reconstitution).

In one embodiment the energy density of the first composition is higher than the energy density of the second composition. In one embodiment it is higher than the energy density of the second and third compositions.

In one embodiment the energy density of the second composition is higher than the energy density of the third composition. In one embodiment it is higher than the energy density of the optional 4^{th} and/or of the optional 5^{th} composition and/or optional 6^{th} composition

In one embodiment the energy density of the third composition is equal to the energy density of the optional 4^{th} and/or optional 5^{th} and/or optional 6^{th} composition.

In one embodiment the energy density of the optional fourth and/or fifth and/or sixth compositions is lower than the energy density of the first and/or second compositions.

It is believed that the strict control of the energy intake over time can lead to health benefits later in life. As such the solution of the proposed invention is particularly tailored to (a) provide sufficient energy for growth, (b) while lowering the energy density intake after the first month to avoid overgrowth (c) while somewhat lowering the energy intake after 2^{nd} months to avoid overgrowth, (d) maintaining then a about constant energy density in the 3^{rd}, 4^{th} and 5^{th} compositions in order to take into account the complementary food supplied to the babies. The energy density of the compositions and their evolution over age of the infant are adapted/tailored to take into account the increasing amount of nutritional composition consumed by the infant.

It is believed that the complementary food is a nutritional factor that has been so far under-accounted for in the typical nutritional schemes.

In one embodiment the array of compositions of the invention are
wherein the energy density (in kcal/100 ml once reconstituted) of the first composition is higher than the energy density of the second composition, and
wherein the energy density (in kcal/100 ml once reconstituted) of the second composition is higher than the energy density of the third composition, and
optionally wherein the energy density (in kcal/100 ml once reconstituted) of the third composition is higher or equal to the energy density of the optional fourth and/or fifth and/or sixth infant composition

### Fat/Lipids

The fat in the set of nutritional compositions may be selected from milk and/or vegetable fat. Typical vegetable fats include palm olein, high oleic sunflower oil, high oleic safflower oil or any mixtures thereof. The fats are a source of long-chain polyunsaturated fatty acids (LC-PUFA). LC-PUFA's have been linked to benefits in infant/young child development. Preferably, the LC-PUFA are selected from docosahexaenoic acid (DHA), arachidonic acid (ARA) or any mixtures thereof. Most preferably, the first, second and third nutritional compositions comprise a mixture of DHA and ARA. Most preferably, the fourth and fifth compositions comprise docosahexaenoic acid DHA only. Where both DHA and ARA are present it is preferable that each composition comprises from 0.14 to 0.16 g/100 g powder of DHA and 0.14 to 0.15 g/100 g powder of ARA.

In some embodiments the array of compositions of the invention are
wherein the fat content (in g fat/100 kcal) of the first composition is higher than the fat content (in g fat/100 kcal) of the second composition, and
wherein the fat content (in g fat/100 kcal) of the second composition is higher than the fat content (in g fat/100 kcal) of the third composition, and
optionally wherein the fat content (in g fat/100 kcal) of the third composition is lower than fat content (in g fat/100 kcal) of the optional fourth infant composition.

In one embodiment the fat content (in g fat/100 kcal) of the fifth composition is equal of higher than the fat content of the fourth composition.

In one embodiment the fat content (in g fat/100 kcal) of the sixth composition is equal of lower than the fat content of the fifth composition and/or 4^{th} and/or 3^{rd} and/or 2^{nd} and/or 1^{st} compositions.

It appears important to the inventors to reduce drastically the fat content of the 6^{th} compositions to take into account the complementary food consumed by the baby at that age.

It appears important to the inventors to increase the fat content of the 4^{th} compositions to take into account the increasing needs of the baby at that age. At progressive decrease during the 2^{nd} and 3 to 6^{th} months (2^{nd} and 3^{rd} compositions) is foreseen to avoid overfeeding fat nutrients (in particular as the overall quantity of composition fed to the infant increases over age).

In one embodiment, the fat content of the first composition is preferably between 48 and 54%, more preferably between 50 and 52% of the total energy for the first composition.

In one embodiment the fat content of the second composition is preferably between 48 and 54%, more preferably between 50 and 54% of the total energy for the second composition.

In one embodiment the fat content of the third composition is preferably between 48 and 54%, more preferably between 50 and 54% of the total energy for the third composition.

In one embodiment the fat content of the fourth (optional) composition is preferably between 35 and 45%, more preferably between 35 and 40% of the total energy for the fourth composition.

In one embodiment the fat content of the fifth (optional) composition is preferably between 35 and 45%, more preferably between 40 and 45% of the total energy for the fifth composition.

In one embodiment of the invention the fat content of the optional fourth and fifth composition is lower (in absolute value and/or as % of the total energy) than the fat content of the first three compositions. The inventors believe that the supply of the adequate decreasing fat content during the first weeks or months (i.e. in the first 3 compositions) is better leveraged when coupled with a higher fat content in the later years (i.e. 4^{th} and 5^{th} compositions) as the needs of the baby increase. In this way the most adequate fat content is delivered over a longer period of time. It is believed that it can be linked to health benefits over time such as lowering the risk of obesity, of cardio vascular diseases, of metabolic syndrome or even diabetes later in life.

In one embodiment the fat content of the optional 6th composition (in absolute value and/or as % of total energy) is lower than the fat content in the 1^{st}, and/or 2^{nd}, and/or 3^{rd} and/or 4^{th} and/or 5^{th} compositions. It is believed that this better takes into account the nutritional boost supplied by the complementary food at that age.

### Carbohydrates

The carbohydrates in the set of nutritional compositions may include lactose, saccharose, maltodextrin, starch and mixtures thereof. In a preferred embodiment, the first and second compositions comprise lactose. Preferably, the amount of lactose in the first and second compositions is between 9.5 and 12 g/100 kcal, preferably between 10 and 11 g/100 kcal. The third and fourth compositions preferably comprise a mixture of lactose and maltodextrin. Preferably, the maltodextrin has a DE of 19. Most preferably, the ratio of lactose to maltodextrin in the third and fourth compositions is 70:30. In one embodiment the carbohydrate source in all compositions comprises or is lactose.

A carbohydrate content of the set of nutritional compositions is as preferably as follows. The carbohydrate content of the first composition is between 35% and 48% of the total energy for said first composition. The carbohydrate content of the second composition is between 40% and 45% of the total energy for said second composition. The carbohydrate content of the third composition is between 42% and 48% of the total energy for said third composition. The carbohydrate content of the optional fourth composition is between 45% and 60% of the total energy for said fourth composition. The carbohydrate content of the optional fifth composition is between 50% and 60% of the total energy for said fifth composition.

It is believed that the relative high carbohydrate content in the 4^{th} and/or 5^{th} composition are best suited to deliver the form of “fast” energy needed at this age, without promoting the fat accumulation.

### Proteins

The proteins may include intact or hydrolysed protein, milk fat globule membrane (MFGM) protein, casein, whey, soy protein, rice proteins or any mixtures thereof.

In a preferred embodiment the 1^{st}, and/or 2^{nd}, and/or 3^{rd} and/or optional 4^{th} compositions (preferably 1^{st} and 2^{nd} and 3^{rd} and optional 4^{th}) are 100% whey proteins, optionally and preferably partially hydrolyzed to provide easier digestibility and lower allergic potential.

In a preferred embodiment, the optional 5^{th} and/or 6^{th} (preferably both) composition comprise a mixture of whey and casein.

Preferably the optional 5^{th} and 6^{th} compositions are intact (non hydrolyzed).

In embodiments of the invention the protein content (expressed in g protein/100 kcal) are
wherein the protein content (in g protein/100 kcal) of said first composition is higher than the protein content of said second composition, and
wherein the protein content (in g protein/100 kcal) of said second composition is higher or equal to the protein content of said third composition, and
optionally wherein the protein content (in g protein/100 kcal) of said third composition is higher or equal to the protein content of said optional fourth and/or optional fifth infant composition.

Optionally the protein content of the optional 6^{th} composition is higher than the protein content of the optional 5^{th} composition.

The protein content in the compositions preferably varies between 1.5 to 2.5 g/100 kcal. For instance, the first composition may comprise a protein content of above 1.8 to 2.25 g/100 kcal. The second composition may comprise a protein content of 1.8 g/100 kcal or 2.0 g/100 kcal (as long as the value is lower than the one of the 1^{st} composition). The third composition may comprise a protein content of 2 g/100 kcal. The protein content of the optional fourth composition is preferably 2 g/100 kcal. The protein content of the optional fifth composition is preferably 2 g/100 kcal. The protein content of the optional sixth composition is preferably 2.2 g/100 kcal.

The compositions are adapted to meet the evolving nutrient requirements of infants and young children. A protein density and/or protein content and/or fat content of the compositions change in the various age groups, to mimic the evolution of human breast milk. In one embodiment the protein density of the compositions is highest during the first month when growth is fastest, and decreases (or remains stable at a relatively lower level compared to the 1^{st} composition) until the 6th month of life. The protein density of the compositions then remains constant and increases again after the second year of life. Especially for older age groups (1 to 2 and 2 to 3 years). The protein density of the compositions can be significantly lower than that what is naturally found in cow's milk (5 g/100 kcal) in order to avoid protein excess. It is believed that a control of the protein density of the compositions helps maintaining the infant in normal growth curves and has effect later in life for the reduction of obesity and excess weight.

Whey protein: The whey protein may be modified sweet whey. Sweet whey is a readily available by-product of cheese making and is frequently used in the manufacture of infant formulas based on cows' milk. However, sweet whey includes a component which is undesirably rich in threonine and poor in tryptophan called caseino-glyco-macropeptide (CGMP). Removal of the CGMP from sweet whey results in a protein with a threonine content closer to that of human milk. This modified sweet whey may then be supplemented with those amino acids in respect of which it has a low content (principally histidine and tryptophan). A process for removing CGMP from sweet whey is described in EP 880902 and an infant formula based on this modified sweet whey is described in WO 01/11990.

The proteins may be intact or hydrolysed or a mixture of intact and hydrolysed proteins. It may be desirable to supply partially hydrolysed proteins (degree of hydrolysis between 2 and 20%), for example for infants believed to be at risk of developing cows' milk allergy. If hydrolysed proteins are required, the hydrolysis process may be carried out as desired and as is known in the art. For example, a whey protein hydrolysate may be prepared by enzymatically hydrolyzing the whey fraction in two steps as described in EP 322589. For an extensively hydrolysed protein, the whey proteins may be subjected to triple hydrolysis using Alcalase 2.4 L (EC 940459), then Neutrase 0.5 L (obtainable from Novo Nordisk Ferment AG) and then pancreatin at 55° C. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

### Optional Whey/Casein Ratio

Preferably, the ratio of whey to casein in the fifth and 6^{th} compositions is 70:30.

When the 1^{st}, 2^{nd} and 3^{rd} compositions comprise a mixture of whey and casein, the second and third compositions can have a protein ratio of whey:casein of 50:50. Preferably, the optional fourth composition has a whey to casein protein ratio of 40:60.

In one embodiment the invention extends to an array of age-tailored compositions comprising:

A first composition having a protein source comprising whey and optionally casein proteins and having a whey:casein ratio between 100:0 and 60:40 and a protein content between 2.0 and 3.0 g protein/100 kcal

In different one embodiment, it also has second composition having a protein source comprising whey and casein proteins and having a whey:casein ratio between 70:30 and 50:50 and a protein content between 1.8 and 2.0 g protein/100 kcal, with the proviso that either the protein content or the whey:casein ratio of the second formula or both (preferably both) is/are lower than for the first formula.

In one embodiment the optional 5^{th} and/or 6^{th} compositions has a protein source comprising whey and casein proteins and have a whey:casein ratio between 70:30 and 50:50 and a protein content between 1.7 and 2.2 g protein/100 kcal with the proviso that either the protein content or the whey:casein ratio of the fifth and/or sixth compositions or both (preferably both) is/are lower than for the first formula.

In other embodiments the first composition has a protein source with a whey:casein ration between 80:20 and 60:40 and a protein content between 2.0 and 3.0 g protein/100 kcal. The second composition have a whey:casein ratio between 70:30 and 50:50 and a protein content between 1.8 and 2.0 g protein/100 kcal.

A third composition can have a whey:casein ratio between 70:30 and 50:50 and a protein content between 1.8 and 2.0 g protein/100 kcal wherein either the protein content or the whey:casein ratio of the second formula or both 8preferably both) is/are lower than for the second formula.

Vitamins, minerals, trace elements and other ingredients Optionally, the compositions may comprise vitamins selected from vitamin A, beta-carotene, vitamin D, vitamin E, vitamin K1, vitamin C, vitamin B1, vitamin B2, niacin, vitamin B6, folic acid, pantothenic acid, vitamin B12, biotin, choline, inositol, taurine, carnitine or any mixtures thereof.

Additionally, the compositions may comprise minerals selected from sodium, potassium, chloride, calcium, phosphorus, magnesium, manganese or any mixtures thereof.

Trace elements such as iron, iodine, copper, zinc, selenium, fluorine, chromium, molybdenum or any mixtures thereof may also be present in the compositions forming the set of nutritional compositions.

The compositions may contain other beneficial substances. The beneficial substances can be nucleotides and/or nucleosides. Nucleotides may be selected from cytidine monophosphate (CMP), uridine monophosphate (UMP), adenosine monophosphate (AMP), guanosine monophosphate (GMP) or any mixtures thereof.

Iron: In one embodiment the iron contents (in mg/100 kcal) is about similar in the first, second, third and optional fourth compositions (i.e. equal value +/−10%).

In one embodiment the iron content of the optional fifth compositions and/or of the optional sixth composition is higher than in the first composition or than in the third composition.

In one embodiment the iron content of the first, second and/or third compositions is between 0.70 and 0.80 mg/100 kcal.

In one embodiment the iron content of the optional fifth and/or sixth compositions is between 1.20 and 1.40 mg/100 kcal.

### Lactoferrin

In one embodiment, any of the compositions forming part of the array may comprise lactoferrin. The lactoferrin may be “carried-over” from the other ingredients of the compositions (such as the protein source) or may be added as a separate ingredient.

The nutritional compositions for use in the present invention may also be supplemented with the bioactive whey protein lactoferrin. Lactoferrin is known inter alia to promote the growth and maturation of the gastrointestinal tract in newborn infants. The lactoferrin content of infant formulas for use in the present invention preferably decreases with increasing age of the infant and counts as part of the protein for the purposes of assessing the protein content of the formula and as part of the whey proteins for the purposes of calculating the whey:casein ratio of the formula. The lactoferrin content of infant formulas for use in the invention is preferably between 1.5 and 0.1 grams/litre, more preferably between 1.0 and 0.3 grams/litre (and/or correspond values for powder compositions).

Preferably the first composition comprises lactoferrin. Most preferably the second composition comprises no or a lower amount of lactoferrin.

### Prebiotics

The composition of the invention can further comprise at least one or one further prebiotic, usually in an amount between 0.3 and 10% by weight of composition.

Prebiotics are usually non-digestible in the sense that they are not broken down and absorbed in the stomach or small intestine and thus remain intact when they pass into the colon where they are selectively fermented by the beneficial bacteria. Prebiotics are preferably added to the compositions of the later age groups (1 to 2 years and 2 to 3 years). In this instance the prebiotics reinforce the immune system, improve gastrointestinal comfort, and prevent discomfort that may arise from diarrhoea.

The composition according to the invention can comprise, in some embodiments, Oligofructose (OF). An example of such OF is the commercial ingredient ORAFTI® by Beneo GmbH (Mannheim, Germany).

In some embodiments the prebiotics of the composition of the invention, comprise other fructooligosaccharides (FOS) or/and galactooligosaccharides (GOS). A combination of prebiotics may be used such as 90% GOS with 10% short chain fructo-oligosaccharides such as in the product by BENEO-Orafti sold under the trademark “Orafti® oligofructose” (see http://www.beneo-orafti.com/Our-Products/Oligofructose) (previously Raftilose) or 10% inulin such as in the product sold by BENEO-Orafti under the trademark “Orafti® inulin” (see http://www.beneo-orafti.com/Our-Products/Inulin) (previously Raftiline®). Another combination of prebiotics is 70% short chain fructo-oligosaccharides and 30% inulin, which is a product sold by BENEO-Orafti® under the trademark “Prebio 1”.

In one embodiment the nutritional composition according the invention comprises a prebiotic selected from the list bovine milk oligosaccharides, inulin, xylooligosaccharides, polydextrose or any combination thereof.

In one embodiment the nutritional composition according the invention comprises a bovine milk oligosaccharide, said bovine milk oligosaccharides being an N-acetylated oligosaccharide, a galacto-oligosaccharide, a sialylated oligosaccharide, or a combination thereof. Such oligosaccharides that may be comprised in the compositions of the invention can be fucosylated oligosaccharides.

A particularly preferred prebiotic is a mixture of galacto-oligosaccharide(s), N-acetylated oligosaccharide(s) and sialylated oligosaccharide(s) in which the N-acetylated oligosaccharide(s) comprise 0.5 to 4.0% of the oligosaccharide mixture, the galacto-oligosaccharide(s) comprise 92.0 to 98.5% of the oligosaccharide mixture and the sialylated oligosaccharide(s) comprise 1.0 to 4.0% of the oligosaccharide mixture. This mixture is hereinafter referred to as “CMOS-GOS”.

Preferably, any of the compositions of the set for the invention contain from 2.5 to 15.0 wt % CMOS-GOS on a dry matter basis with the proviso that the composition comprises at least 0.02 wt % of an N-acetylated oligosaccharide, at least 2.0 wt % of a galacto-oligosaccharide and at least 0.04 wt % of a sialylated oligosaccharide.

Suitable galacto-oligosaccharides to be optionally part of the compositions of the invention include Galβl,6Gal, Galβl,6Galβl,4Glc, Galβl,6Galβl,6Glc, Galβl,3Galβl,3Glc, Galβl,3Galβl,4Glc, Galβ1,6Galβ1,6Galβ1,4Glc, Galβ1,6Galβ1,3Galβ1,4Glc, Galβ1,3Galβ1,6Galβ1,4Glc, Galβl, 3Galβl,3Galβl,4Glc, Galβl,4Galβl, 4Glc and Galβl,4Galβl, 4Galβl,4Glc.

Synthesised galacto-oligosaccharides such as Galβl,6Galβl, 4Glc, Galβl,6Galβl,6Glc, Galβl,3Galβl,4Glc, Galβl,6Galβl,6Galβl,4Glc, Galβl,6Galβl,3Galβl,4Glc and Galβl,3Galβl,6Galβl,4Glc, Galβl,4Galβl,4Glc and Galβl,4Galβl,4Galβl,4Glc and mixtures thereof are commercially available under the trademarks Vivinal® and Elix'or®. Other suppliers of oligosaccharides are Dextra Laboratories, Sigma-Aldrich Chemie GmbH and Kyowa Hakko Kogyo Co., Ltd. Alternatively, specific glycosyltransferases, such as galactosyltransferases may be used to produce neutral oligosaccharides. Suitable sialylaled oligosaccharides include NeuAcα2, 3Galβl, 4Glc and NeuAcα2, 6Galβl, 4Glc. These sialylated oligosaccharides may be isolated by chromatographic or filtration technology from a natural source such as animal milks. Alternatively, they may also be produced by biotechnology using specific sialyltransferases either by enzyme based fermentation technology (recombinant or natural enzymes) or by microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP) from DP=1 onwards.

The prebiotics are preferably present in the compositions in an amount 1 to 20 wt %, preferably 2 to 15 wt % on a dry matter basis.

In one embodiment the prebiotics present in the first, second and optionally third compositions are different (in nature and/or amount).

### Human Milk Oligosaccharides:

Human milk oligosaccharides (HMOs) are, collectively, the third largest solid constituents in human milk, after lactose and fat. HMO usually consists of lactose at the reducing end with a carbohydrate core that often contains a fucose or a sialic acid at the non-reducing end. There are approximately one hundred milk oligosaccharides that have been isolated and characterized, however these represent only a very small portion of the total number remaining to be characterized.

In the past, infant formulae were developed using HMO ingredients, such as fucosylated oligosaccharides, lacto-N-tetraose, lacto-N-neotetraose, or sialylated oligosaccharides. The composition of the invention may contain 2′-fucosyllactose (2FL) and/or a N-acetyl-lactosamine such as lacto-N-neotetraose (LNnT) or lacto-N-tetraose (LNT).

In one embodiment the nutritional composition according the invention comprises human milk oligosaccharide selected from the list consisting of N-acetyl-lactosamine, sialylated oligosaccharides, fucosylated oligosaccharides, 2FL, LNnT, LNT or a combination thereof.

N-acetyl-lactosamine: In some embodiments the composition of the invention contains at least one N-acetyl-lactosamine. That is to say that the composition according to the invention contains N-acetyl-lactosamine and/or an oligosaccharide containing N-acetyl-lactosamine. Suitable oligosaccharides containing N-acetyl-lactosamine include lacto-N-tetraose (LNT) and lacto-N-neotetraose (LNnT).

Thus, according to the invention, the N-acetyl-lactosamine is preferably selected from the group comprising lacto-N-tetraose (LNT) and lacto-N-neotetraose (LNnT).

Preferably the composition according to the invention contains from 0.1 to 3 g N-acetyl-lactosamine per 100 g of composition on a dry weight basis. Preferably it contains 0.1 to 3 g of LNnT per 100 g of composition on a dry weight basis.

In one embodiment the nutritional composition according the invention comprises a N-acetyl-lactosamine, preferably selected from the group comprising lacto-N-tetraose (LNT) and lacto-N-neotetraose (LNnT).

Sialylated Oligosaccharides: The composition according to the invention, in some embodiments, can comprise one or more sialylated oligosaccharides.

The sialylated oligosaccharides may be selected from the group comprising 3′-sialyllactose and 6′-sialyllactose. Preferably, both 3′-sialyllactose and 6′-sialyllactose are present in said composition. In this embodiment, the ratio between 3′-sialyllactose and 6′-sialyllactose lies preferably in the range between 5:1 and 1:2.

Preferably the composition according to the invention contains from 0.05 to 2 g, more preferably 0.1 to 2 g, of sialylated oligosaccharide(s) per 100 g of composition on a dry weight basis.

In one embodiment the nutritional composition according the invention comprises sialylated oligosaccharide, preferably selected from the group comprising 3′-sialyllactose and 6′-sialyllactose. More preferably said composition comprises both 3′-sialyllactose and 6′-sialyllactose, the ratio between 3′-sialyllactose and 6′-sialyllactose lying preferably in the range between 5:1 and 1:2.

Fucosylated oligosaccharide: The composition according to the invention may comprise one or more fucosylated oligosaccharides. Preferably the fucosylated oligosaccharides consist or comprises 2′-fucosyllactose (2-FL).

The fucosylated oligosaccharide may be selected from the group comprising 2′-fucosyllactose, 3-fucosyllactose, difucosyllactose (DiFL), lacto-N-fucopentaoses (that is to say acto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. A particularly preferred fucosylated oligosaccharide is 2′-fucosyllactose (2-FL) or DiFL.

Preferably, the composition according to the invention contains from 0.1 to 3 g of fucosylated oligosaccharide(s) per 100 g of composition on a dry weight basis, most preferably being 2FL

In one embodiment the nutritional composition according the invention comprises a fucosylated oligosaccharide, preferably selected from the group comprising 2′-fucosyllactose, 3-fucosyllactose, difucosyllactose, lacto-N-fucopentaoses (that is to say acto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II, and preferably the fucosylated oligosaccharide is 2′-fucosyllactose (2-FL).

### Probiotics

The composition of the invention can further comprise at least one probiotic bacterial strain, said probiotic bacterial strain preferably being Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus 30 johnsonii CNCM 1-1225, Streptococcus salivarius DSM 13084 sold by BLIS Technologies Limited of New Zealand under the designation KI2, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

Preferably, the composition according to the invention contains from 10e3 to 10e12 cfu of probiotic bacterial strain, more preferably between 10e7 and 10e12 cfu, per g of composition on a dry weight basis.

In one embodiment the nutritional composition of the comprises a probiotic bacterial strain selected from the list consisting of Lactobacillus acidophilus, Lactobacillus salivarius, Lactobacillus rhamnosus, Lactobacillus paracasei, Lactobacillus casei, Lactobacillus johnsonii, Lactobacillus plantarum, Lactobacillus fermentum, Lactobacillus lactis, Lactobacillus delbrueckii, Lactobacillus helveticus, Lactobacillus bulgari, Lactococcus lactis, Lactococcus diacetylactis, Lactococcus cremoris, Streptococcus salivarius, Streptococcus thermophilus, Bifidobacterium lactis, Bifidobacterium animalis, Bifidobacterium longum, Bifidobacterium breve, Bifidobacterium infantis, or Bifidobacterium adolescentis or any mixture thereof. Most preferably, the probiotic is Bifidobacterium lactis. In one embodiment the probiotic comprises L. Reuteri.

Any of the nutritional compositions or all may also comprise at least one probiotic bacterial strain.

In one embodiment only the first and second composition comprises probiotics.

The probiotics establish a healthy gut microbiota and strengthen natural immune defenses. The probiotics also stimulate a development of the immune system at introduction of weaning food and prevent diarrhea.

The amount of probiotic, if present, likewise preferably varies as a function of the age of infants and young children. Generally speaking, the probiotic content may increase with increasing age of the infant for example from 10³ to 10¹² cfu/g composition, more preferably between 10⁴ and 10⁸ cfu/g composition (dry weight). In a preferred embodiment, any of the nutritional compositions of the set comprise 2×10⁷ cfu/g or 2×10⁸ cfu/g.

In one embodiment the first, second, and optionally third compositions comprise different probiotics. In is foreseen that various probiotics can have age-tailored effect and adapting such probiotics to the intended age of the composition provides further effects.

### Preparation of the Nutritional Compositions

The nutritional compositions may be prepared in any suitable manner. For example, an infant formula may be prepared by blending together a protein source, a carbohydrate source, and a fat source in appropriate proportions. If used, emulsifiers may be included in the blend. Any additional vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger. The liquid mixture may then be cooled to about 60° C. to about 85° C. for example by flash cooling. The liquid mixture may then be homogenised for example in two stages at about 7 MPa to about 40 MPa in the first stage and about 2 MPa to about 14 MPa in the second stage. The homogenised mixture may then be further cooled to add any heat sensitive components such as vitamins and minerals. The pH and solids content of the homogenised mixture are conveniently standardised at this point.

The homogenised mixture is transferred to a suitable drying apparatus such as a spray drier or freeze drier and converted to powder. The powder should have a moisture content of less than about 3% by weight. Alternatively, the homogenised mixture is concentrated.

If it is desired to add probiotic(s), they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the powdered infant formula by dry mixing.

### Packing

In an embodiment of the invention, the nutritional compositions are packed in single dose units. Each single dose unit comprises sufficient nutritional composition to prepare a single serving upon reconstitution with water.

A serving typically provides an infant or young child with 65 or 100 to 200 kcal. Thus, a single serving generally comprises between 11 and 30 g of powder to be reconstituted with water. Alternatively, if the nutritional composition is a concentrate, a single serving includes 30 to 70 mL of concentrate to be diluted with 100 ml to 200 ml of water.

### Kit

The invention also pertains to an age-tailored kit for infants and young children. The kit comprises the set of nutritional compositions as described herein. The nutritional compositions are packed in single dose units as mentioned. The single dose units may be in the form of stick packs or sachets.

The single dose units may be disposable capsules equipped with opening means contained within the capsule to permit draining of the reconstituted formula directly from the capsule into a receiving vessel such as a bottle. Such a method of using capsules for dispensing an infant or young child nutritional composition is described in WO2006/077259. The different nutritional compositions forming part of the set of the invention may be packed into individual capsules and presented to the consumer in multipacks containing a sufficient number of capsules to meet the requirements of infants and young children one week for example. Suitable capsule constructions are disclosed in WO2003/059778.

All the nutritional compositions described herein can be made part of a kit according to the invention.

### Feeding Regimen

The set of the invention is also used for providing infants and young children with a balanced nutritional diet for at least 6 months or the first two years of life.

Preferably the set of the invention promotes the compliance of the care-givers to the nutritional scheme proposed by the present invention, along an extended period of time (e.g. 2 years) in order to capitalize the long term health benefits recited therein. Preferably, and most importantly, however the care-givers must be provided with the choice and information for varying the diet of the infants according to their specific needs, perceived needs or medical conditions.

Another facet of the invention therefore relates to an infants and young children nutrition regimen. The regimen comprises feeding an infant the set of nutritional compositions at specific ages as mentioned already

An example of the feeding regimen according to the invention, and using the compositions as capsules is shown below.

| | | Feed |
|---|---|---|
| Infant Age | Quantity per feed | per day |
| 1^{st} month | 1 capsule/90 ml | 5-7 |
| 2nd Month or | 1 capsule/150 ml | 4-6 |
| 2nd Month | 1 capsule/180 ml | 3-5 |
| 3rd to 6th Month, or | 1 capsule/180 ml | 3-5 |
| 3rd to 6th Month | 1 capsule/210 ml | 2-4 |
| 7th to 12th Month | 1 capsule/240 ml | 2-3 |
| 13-24^{th} month | 1 capsule/240 ml | 2-3 |
| 25^{th}-36^{th} months | 1 capsule/240 ml | 2-3 |

Complementary foods can also be taken with the set of nutritional compositions. The complimentary food may be any of the foods available for the corresponding age range. These complimentary foods include pureed vegetables, meats, fish, fruits, etc.

It has been found that such a regimen provides a child with a balanced nutritional intake at least for the first two years of life and has long-term health benefits on the infant/young child later in life.

### Health Benefits/Prevention of Undesired Health Condition

It has been found that array of nutritional compositions when administered to infants and young children provides short, mid and long-term benefits by avoiding and/or preventing and/or reducing the risk of sub-optimal body composition.

In particular, the beneficial effects can include the reduction or prevention of over-weight or obesity later in life, the reducing of cardiovascular diseases, the reduction or prevention of diabetes or diabetes-related health condition. The reduction or prevention can be a reduction of the risk of occurrence and/or reduction of the severity of events, and/or reduction of the frequency of events.

Other health benefits associated with the set of nutritional compositions include a growth within the usually accepted growth curves (from weight gain or size growth or combination thereof), the reduction of occurrence of diabetes, especially type II diabetes and better immune status (including less bacterial and/or viral infections), and/or less allergies.

Further benefits also include the reduction of cardiovascular diseases later in life, hypertension and renal dysfunction.

The beneficial health effects can be measured for their reduced frequency of occurrence and/or for their attenuated symptoms when they occur and/or their low negative health impact (i.e. intensity of effect). This is defined in comparison to the average occurrence/frequency/intensity of the health effects/status in the general population. Comparison are most adequate when the reference group is a population of same or similar genetic/ethnic origins.

In the present document the terms “sub-optimal body composition”, “unhealthy body composition” and “un-adequate body composition” are used interchangeably with the same meaning.

The sub-optimal body composition (also called unhealthy body composition or un-adequate body composition), can preferably refer to or be defined as the fat-mass and/or fat-free mass of the subject (i.e. the fat balance of the body composition). Similarly to the rate of development of the infants (growth curves; measuring the weight and/or the size in the infants/young children), these body composition parameters are indeed also of significant importance. In some embodiment the “sub-optimal body composition” also encompass or may be defined by the suboptimal body mass, body volume, body density, and weight gain patterns. In one preferred embodiment the body composition is the fat-mass and/or the fat-free mass balance.

The body composition is qualified as sub-optimal (also called unhealthy body composition or un-adequate body composition), when the measured parameters defining the body composition (e.g. fat mass and/or fat-free mass) are significantly different from those of exclusively breast-fed infants (and/or infants having received a majority of their caloric input as human breast milk during a significant period of time, preferably 4 or 6 months or more). The comparison is made on a test group or population (i.e. multiple subjects) and the statistical significance is observed. In one embodiment the statistical test is made at a p value of 0.01, in another embodiment of 0.05 or with a p value (or similar statistical test) scientifically relevant.

Sub-optimal body composition is in general highly undesirable and represents a health status that is not ideal for an infant. It underlays a clearly undesired health condition for the infant both short term, mid term and longer term.

Un-adequate or sub-optimal fat-mass and/or fat-free mass and suboptimal feeding associated body mass and weight gain patterns have indeed been linked to negative health effects later in life (such as diabetes, obesity and/or cardiovascular diseases). See references (Koontz et al., longitudinal changes in infant body composition: association with childhood obesity. Pediatric Obesity 2014 9 9 (6) e141-e144. Dietz, Health Consequences of Obesity in Youth: Childhood Predictors of Adult Disease. Pediatrics 1998; 101:518-525. Osmond et al., et al, Infant, and Childhood Growth Are Predictors of Coronary Heart Disease, Diabetes, and Hypertension in Adult Men and Women. Environmental Health Perspectives 2000; 108, Supplement 3.)

For determining what should be considered as desirable a body composition, comparison are made to the body composition (at same age of infants and/or young children who are exclusively or primarily breast-fed during a significant period of time (for example first 6, 8 or first 12 months of life). “Primarily breast fed” refers to receiving more than 50% of the energy input from breast feeding.

Some studies have shown that most conventional infant feeding (by synthetic nutritional compositions) may induce a body composition that is different from the body composition of breast-fed infants.

See in particular Gale et al.; effect of breastfeeding compared with formula feeding on infant body composition: a systematic review and meta-analysis. Am J Clin Nutr 2012; 95:656-69.

Gale et al. conducted a systematic review and meta-analysis of more than 10 studies and highlighted the undesirable effect (see Gale et al.; effect of breastfeeding compared with formula feeding on infant body composition: a systematic review and meta-analysis. Am J Clin Nutr 2012; 95:656-69). Specifically, fat mass is reported to be lower in formula-fed (vs. breastfed) infants at age 3-4 mo (month) and 6 mo, and to be higher in formula-fed (vs. breastfed) infants at 12 mo. Fat-free mass is reported to be higher in formula-fed (vs. breastfed) infants at 3-4 mo, 8-9 mo, and 12 mo. Additionally, the percentage of fat mass is reported to be lower in formula-fed (vs. breastfed) infants at 3-4 mo and 6 mo.

The below table shows the key results of one studies (Gale 2012, meta-analysis) showing the comparison between breast fed and formula fed infants as for the fat mass/fat-free mass.

TABLE 1

| Study reference | age | gender | country | N |
|---|---|---|---|---|
| Gale 2012 | 3-4 m | both | pooled | 247BF, |
| Systematic review | | | | 199FF |
| and meta- | | | | |
| analyses | | | | |
| | Fat-free mass | | | |
| | Breast Fed | Formula fed | | |
| Study reference | infants | infants | difference | P-value |
| Gale 2012 | | | 0.13 (0.03, | 0.01 |
| Systematic review | | | 0.23) | |
| and meta- | | | | |
| analyses | | | | |
| Study reference | BC method | | | |
| Gale 2012 | Models ≥2 | | | |
| Systematic review | compartments | | | |
| and meta- | (exclude | | | |
| analyses | skinfold) | | | |
| | Fat mass | | | |
| | Breast Fed | Formula fed | | |
| Study reference | infants | infants | difference | P-value |
| Gale 2012 | | | 0.09 (−0.18, | 0.04 |
| Systematic review | | | −0.01) | |
| and meta-analyses | | | | |

Such effect may be attributed to various factors such as the synthetic nature of the ingredients, the “incompleteness” of the recipes which can only mimic Human Breast Milk (HBM) incompletely, the lack of various bioactive factors in the synthetic nutritional compositions, the different amounts of nutrients (compared to HBM), the feeding habits induced by the care givers, the mode of delivery of the synthetic nutritional compositions (such as baby bottles, teats, etc. . . . ). Little is known on the factors and how they interplay together.

Hence designing and proposing a solution to avoid the undesired effect on sub-optimal body composition generally induced by conventional infant formula was an unresolved problem before the present invention.

By carefully selecting the proper ingredients and their proper amounts in the array of compositions of the present invention, by targeting these ingredients are specific age of the infant/young children, by varying the ingredients (in terms of nature and/or amount) over the age of the infant/young children in a particular manner (age tailored compositions), by splitting the array of compositions into more than the 2 conventional ages (conventional stage 1 and stage 2), the inventors have arrived to an optimized array of compositions that prevents/avoid the risk of sub-optimal body compositions and related later-in-life sub-optimal conditions. The inventors could design, construct and execute a pertinent clinical study demonstrating the effect (see experimental data).

### Later in Life

While the effect on body composition can be measured short-term or mid-term, after or during the nutritional intervention, the long term health effects and/or the effect “later in life” are evidenced at a certain time after the diet of the invention has been discontinued.

Typically such beneficial health effects are expected to be seen (scientifically measurable) 1, 2, 5, 7, 10, 15, or 20 years after having stopped the claimed set of compositions. In one embodiment these times are the time when the health effects start to the observed and the health effect continue to be measurable for a period of 1, 3, 5, 10, 15, 20, 30 additional years.

The health effects can hence be measurable at the age of 2, 3, 4, 5, 7, 10, 13, 15, 18, 20, 25, 30, 35, 40, 45, 50, or 60. In one embodiment the health effects can be measurable from these respective ages and for a period of 1, 3, 5, 10, 15, 20, 30 additional years. The health effect is an increase life expectancy of 1 week, 2 weeks, 1 month, 3 months, 6 months, or 1, 2, 3, 4, 5, 10 years in comparison to the relevant general population.

For example the infants receiving the claimed set of compositions can be shown to experience beneficial health effect at the age of 7 or 10 and/or 5 or 7 years after having stopped the claims diet.

It has further been found that the nutritional compositions of the invention work in synergy such that optimal health effects are observed when the nutritional compositions are used consequently (and/or sequentially). Therefore, using the nutritional compositions independently (i.e. not as part of the array of nutritional compositions) would not achieve the beneficial effects to the same extent. One can understand that the long term effect is the set of nutritional compositions is better capitalized when the individual compositions are used over a long period of time (for example during at least the first 2 or 3 years of life) and on a regular basis for example when the use of the composition of the invention cover 30% or more, 50% or more, 75% or more 90% or more, 95% or more of the daily caloric intake of the child. The long term effect can be evidenced by measuring parameters such as the body weight (and/or growth curve by mass), the body size, occurrence (frequency) of obesity-related conditions or diseases and the like.

### Metabolite Markers

The relationship between diet, feeding, growth and body composition is complex. On the molecular level, neuropeptides, hormones and cytokines (e.g., ghrelin, IGF-1, insulin, and leptin) all play essential roles by stimulating or suppressing signalling network which has downstream effect on cell growth, cell differentiation, appetite regulation, and ultimately behaviour and health outcomes. Furthermore, the levels of these molecules have been serving as biomarkers to reflect the metabolic health status. Compared to exclusively breast fed infants, formula fed infants are reported to have higher levels of insulin and/or IGF-1 at early life. In the “Early Protein Hypothesis”, it is proposed that these markers play a role in programing further risks of obesity and associated disorders.

In one embodiment health effect of the array of nutritional compositions of the invention is accompanied by C-peptide, grelin, IGF-1, insulin, and/or leptin levels that are equal or similar (i.e. equal +/−15%) to the levels exhibited by infants that have been exclusively breast-fed during the first 4 months of life. This was surprisingly identified by the inventors as being associated with the array of compositions of the invention whereas as difference metabolite levels were expected (as for conventional infant formula).

### Experimental Data/Clinical Study:

The inventors have study the field and could arrive to the present invention by designing, setting up, running and analysing the following experimental study, which results provided unexpected outcome.

It has been surprisingly found that providing the nutritional compositions of the invention to infants/young children induce the prevention of undesired health effects such as the build up of suboptimal body composition. Such sub-optimal body composition, in terms of mass-fat and/or fat-free mass is conventionally observed when comparing formula fed infants to breast fed infants; see Gale et al.; effect of breastfeeding compared with formula feeding on infant body composition: a systematic review and meta-analysis. Am J Clin Nutr 2012; 95:656-69.

### Clinical Study Set Up

The study was a one-arm (n=66) 12-month prospective open-label study of infants who received BabyNes (four formulas/nutritional compositions catering to the nutritional needs at 0-1 mo, 1-2 mo, 2-6 mo and 6-12 mo of age, according to the invention and in line with the compositions of example 5). A group of breastfed infants (n=32) was also included as a reference. The study population for the formula-fed group consisted of healthy, full-term newborn male and female infants ages 0 to 21 days at enrollment whose mother voluntarily elected to feed her infant exclusively formula. Infants received exclusive feedings with BabyNes formulas from enrollment through 4 months of age in amounts suitable for their age and appetite. Infants in the breastfed group were 3 months of age at enrollment, had been breastfed exclusively since birth, and would be exclusively breastfed at least through 4 months of age. After 4 months, dietary diversification was allowed for both groups.

The study was conducted in the Children's Hospital of Fudan University in accordance with the Declaration of Helsinki and its subsequent amendments, in conformance with the International Conference on Harmonization (ICH) guidelines for Good Clinical Practice (GCP), and adherence with the applicable regulatory or legal requirements.

The primary objective of the study was to determine whether infants fed with the BabyNes System grow comparable with the WHO 2006 Child Growth Standard at 4 months of age. Key secondary objectives include comparing the body composition at 3 and 4 months and blood markers of metabolic health at 4 and 12 months between BabyNes fed infants and breastfed reference.

Body composition (including body density, body mass, body volume, fat mass, fat-free mass, fat mass percentage, fat-free mass percentage) were measured using a PEA POD® (COSMED) which is an Air Displacement Plethysmography (ADP) system using whole body densitometry to determine body composition. It is the gold standard for non-invasive infant body composition assessment.

Whole blood were drawn from infants and serum were separated for subsequent analysis. Serum metabolic markers (C-peptide, grelin, IGF-1, insulin, and leptin) were measured with commercial ELISA kits according to the manufacturer instructions (for C-peptide: Millipore Catalog No. EZHCP-20K; for ghrelin: Millipore Catalog No. EZGRA-88K; for IGF-1: AdipoBiotech Catalog No. SK00053-02; for insulin: Millipore Catalog No. EZHIASF-14K; for leptin: Millipore Catalog No. EZHL-80SK).

### Clinical Study Results

For body composition, there was no statistically significant difference between BabyNes-fed infants and breastfed reference group (p>0.5) on fat mass percentage, fat-free mass percentage, body mass, body volume or body density at 3 months or 4 months of age.

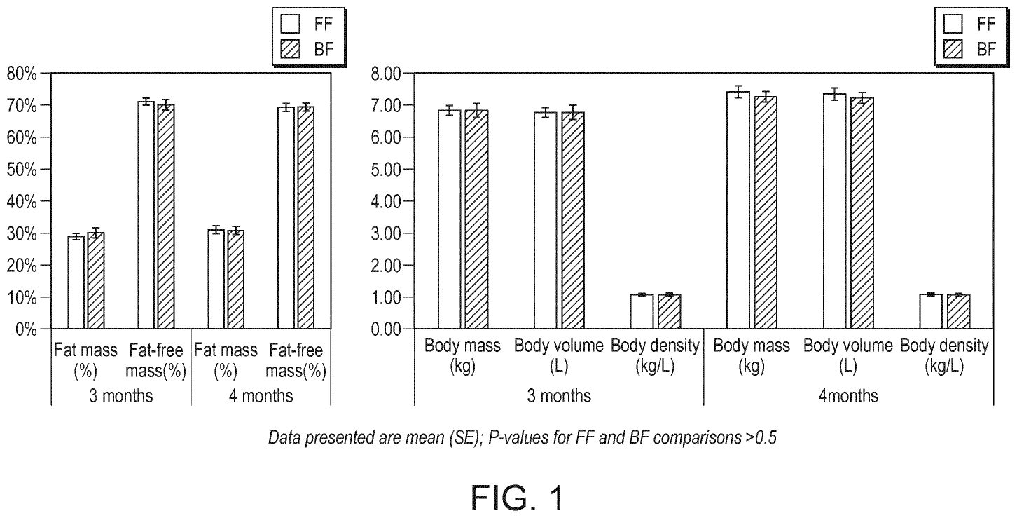

The results are shown in FIG. 1. It shows that BabyNes formula-fed (FF) infants did not statistically differ from the breastfed (BF) reference on fat mass percentage, fat-free mass percentage, body mass, body volume or body density at 3 months or 4 months. The inventors had expected the fat mass parameters to differ between the two groups as they would do with conventional infant formula.

Additionally, there was no statistically significant difference between BabyNes-fed infants and breastfed reference group (p>0.4) for serum blood markers (C-peptide, grelin, IGF-1, insulin, and leptin) at 4 mo of age. Four of five serum markers also did not differ significantly at 12 mo of age (p>0.3) between formula-fed and breastfed infants and only IGF-1 was higher (p=0.035) in formula-fed infants.

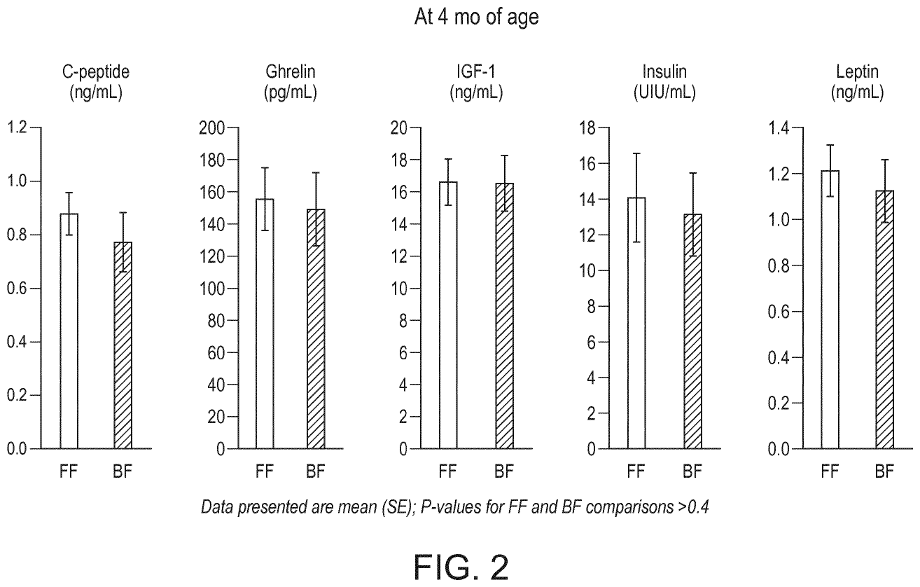

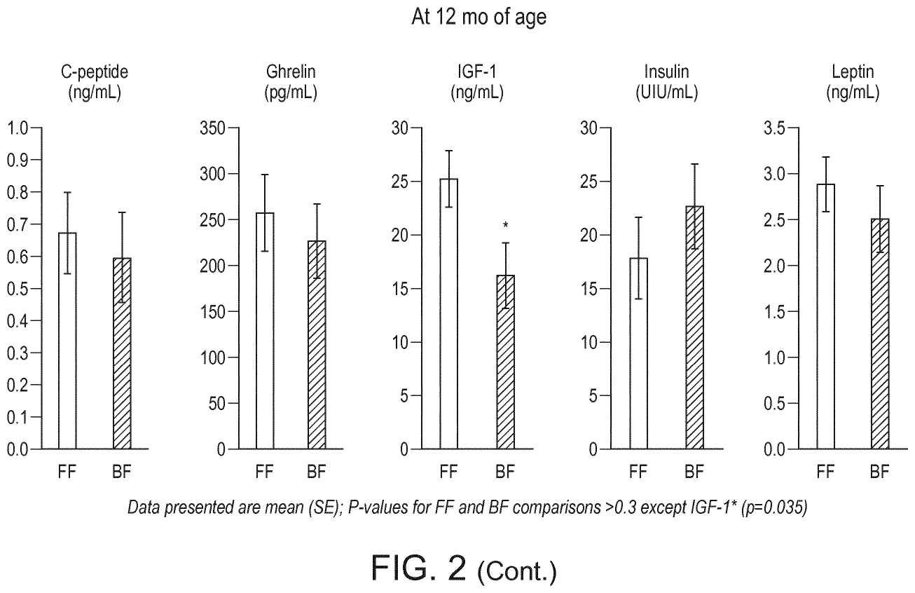

The results are shown in FIG. 2. It shows that at 4 mo (months) of age, formula-fed (FF) infants did not statistically differ from the breastfed (BF) reference on all five serum metabolic markers (C-peptide, grelin, IGF-1, insulin, and leptin); at 12 mo of age, BabyNes formula-fed (FF) infants did not statistically differ from the breastfed (BF) reference on four serum metabolic markers (C-peptide, grelin, insulin, and leptin) but had higher IGF-1. The inventors had expected the serum metabolic markers to differ between the two groups as they would do with conventional infant formula.

### Example 1

An age tailored array of nutritional compositions according to the invention is exemplified below.

Nutritional compositions 4^{th} and 5^{th} are optional.

### Caloric Content:

| Composition | 1^{st} | 2^{nd} | 3^{rd} | 4^{th} | 5^{th} |
|---|---|---|---|---|---|
| Energy | 67 | 65 | 63 | 63 | 63 |
| Kcal/100 ml | | | | | |

### Protein Content:

| Composition | 1^{st} | 2^{nd} | 3^{rd} | 4^{th} | 5^{th} |
|---|---|---|---|---|---|
| Protein | 14.5- | 12.5- | 11.8 | 11.7- | 12.8- |
| g/L | 15.2 | 13 | | 19.2 | 19.5 |
| Protein | 2.18- | 1.93- | 1.88 | 1.86- | 1.9- |
| g/100Kca1 | 2.26 | 2.01 | | 3.04 | 3.1 |

### Fat Content:

| Composition | 1^{st} | 2^{nd} | 3^{rd} | 4^{th} | 5^{th} |
|---|---|---|---|---|---|
| Fat g/L | 37.8 | 33.6 | 32.1 | 35.3 | 35.3 |
| Fat g/ | 5.64 | 5.19 | 5.11 | 5.61 | 5.61 |
| 100 Kcal | | | | | |

### Carbohydrate Content:

| Composition | 1^{st} | 2^{nd} | 3^{rd} | 4^{th} | 5^{th} |
|---|---|---|---|---|---|
| Carbohydrate | 62-67 | 73.8- | 73 | 58.2- | 58.1- |
| (100% | | 73.3 | | 66.1 | 67.1 |
| lactose) | | | | | |
| Carbohydrate | 10.1 | 11.4 | 11.6 | 9.2- | 9.2- |
| g/100 Kcal | | | | 10.5 | 10.6 |

Whey casein ratio and lactoferrin

| Composition | 1^{st} | 2^{nd} | 3^{rd} | 4^{th} | 5^{th} |
|---|---|---|---|---|---|
| Whey casein | 100/0 | 100/0 | 100/0 | 70/30 | 70/30 |
| ratio | | | | | |
| Lactoferrin | measurable | measurable | measurable | measurable | measurable |
| content | | | | | |
| (wt %) | | | | | |

### Example 2

Another array of nutritional compositions according to the invention is shown in the below.

| | | | | | 4^{th} |
|---|---|---|---|---|---|
| | Composition | 1^{st} | 2^{nd} | 3^{rd} | (optional) |
| Basics | Reconstitution | 100 to 200 | 230 | 230 | 230 |
| | RTD Volume | | | | |
| | (ml) | | | | |
| | Energy | 65 | 64 | 62.5 | 63 |
| | density | | | | |
| | (kcal/100 ml) | | | | |
| Proteins | Content | 1.8- | 1.8 | 2 | 2.25 |
| | (g/100 kca1) | 2.25 | | | |
| | Content (g/l) | 11.3-15.1 | 11.3 | | |
| | Whey:Casein | 70:30 | 50:50 | 50:50 | 40:60 |
| | Functional | . | | | — |
| | Protein | | | | |
| Carbohydrates | Type | Lactose | Lactose | Lactose/MD | Lactose/MD |
| | | | | DE19 | DE19 |
| | | | | (70:30) | (70:30) |
| | Content | 9.7 to 11.6 | 10.6 | 10.6 | 14.2 |
| | (g/100 kcal) | | | | |
| | Content (g/l) | 65.0 to 73.5 | 66.8 | | |
| Lipids | Type | Milk & Veg. | Milk & Veg. | Milk & Veg. | Fat mix |
| | | | | | follows |
| | | | | | AHA: |
| | | | | | sat. |
| | | | | | Fat |
| | | | | | <7% E + |
| | | | | | polyuns. |
| | | | | | <10% E |
| | | | | | LA/ALA |
| | | | | | 5.0 |
| | Content | 5.1 to 5.8 | 5.6 | 5.6 | 4 |
| | (g/100 kcal) | | | | |
| | content (as % | 45.9 to 52.2 | 48% to | 48% to | 32% to |
| | of total | | 52% | 52% | 42% |
| | energy) | | | | |
| | Content (g/l) | 32.1 to 38.9 | 35.3 | | |
| | LC-PUFA | DHA + | DHA + | DHA + | DHA |
| | | ARA | ARA | ARA | 0.3% tfa |
| Soluble Fibers | Content | | | | |
| | (g/100 kcal) | | | | |
| Probiotics | Type | B. lactis | | | |
| | Content | 2 × 10⁷ cfu/g | | | |
| Nucleotides | CMP (mg/100 | 1.1 | — | | — |
| | kcal) | | | | |
| | UMP | 0.7 | — | | — |
| | AMP | 0.7 | — | | — |
| | GMP | 0.2 | — | | — |
| | NucleoPremix | | — | | — |
| Minerals (/100 kcal) | Na (mg) | 25 to 37.5 | 25 | 25 | — |
| | K (mg) | 80 to 95 | 80 | 80 | — |
| | Na/K (molar | 0.53 to 0.67 | 0.53 | 0.53 | — |
| | ratio) | | | | |
| | (Na + K)/Cl | 1.71 to181 | 1.71 | 1.71 | — |
| | molar ratio | | | | |
| | Cl (mg) | 65 to 80 | 65 | 65 | — |
| | Ca (mg) | 60 | 60 | 80 | 80 |
| | P (mg) | 33 | 33 | 50 | 50 |
| | Mg (mg) | 7 | 7 | 10 | 10 |
| | Mn (μg) | 5 | 5 | — | — |
| | Ca/P | 1.8 | 1.8 | 1.6 | 1.6 |
| Vitamins (/100 kcal) | Vit. A (mg RE) | 0.09 to | 0.09 | 0.06 | 0.06 |
| | Beta carotene | 01125 | | | |
| | (μg) | | | | |
| | Vit. D (mg) | 0.0015 | 0.0015 | 0.0018 | 0.0018 |
| | Vit. E (mg) | 1.3 | 1.3 | 1.3 | 1.3 |
| | Vit. K1 (μg) | 8 | 8 | 4 | 4 |
| | Vit. C (mg) | 15 | 15 | 10 | 10 |
| | Vit. B1 (mg) | 0.07 to 0.1 | 0.1 | 0.08 | 0.08 |
| | Vit. B2 (mg) | 0.1 | 0.1 | 0.08 | 0.08 |
| | Niacin (mg) | 0.5 | 0.5 | 0.8 | 0.8 |
| | Vit. B6 (mg) | 0.05 | 0.05 | 0.07 | 0.07 |
| | Folic acid (μg) | 15 to 16 | 15 | 15 | 15 |
| | Pantothenic | 0.7 to 0.8 | 0.8 | 0.4 | 0.4 |
| | Acid (mg) | | | | |
| | Vit. B12 (μg) | 0.2 | 0.2 | 0.15 | 0.15 |
| | Biotin (μg) | 2 | 2 | 1.5 | 1.5 |
| | Choline (mg) | 20 | 20 | 30 | 30 |
| | Inositol (mg) | 25 | 20 | — | — |
| | Taurine (mg) | 8 | 6 | — | — |
| | Carnitine (mg) | 1.5 | — | — | — |
| Trace Elements (/100kcal) | Fe (mg) | 0.7 | 1 | 1 | 1 |
| | I (μg) | 15 to 20 | 20 | 15 | 15 |
| | Cu (mg) | 0.06 to 0.08 | 0.06 | 0.05 | 0.05 |
| | Zn (mg) | 1 to 1.2 | 0.8 | 0.6 | 0.6 |
| | Se (μg) | 3 to 4 | 3 | 3.5 | 3.5 |
| | F (μg) | | — | — | — |

### Example 3

The information provided in example 2 can be combined with the information of example 1. Such combination is incorporated herein as example 3.

### Example 4

A further example of the array of compositions of the invention is provided below:

| | Age Range | | | | | |
|---|---|---|---|---|---|---|
| | | | 1-2 | 3-4 | 5-6 | |
| | 1-2 weeks | 3-4 weeks | months | months | months | 7-12 months |
| Reconstitution | 100 | 130 | 160 | 180 | 200 | 230 |
| volume (ml) | | | | | | |
| Energy density | 63 | 63 | 63 | 63 | 63 | 63 |
| (kcal/ml) | | | | | | |
| Protein content | 2.5 | 2.0 | 2.0 | 1.8 | 1.8 | 1.8 |
| (g/100 kcal) | | | | | | |
| Whey:casein | 70:30 | 60:40 | 60:40 | 60:40 | 60:40 | 50:50 |
| Lactoferrin | 1.0 | 0.5 | 0.5 | 0.3 | 0.3 | |
| (g/l) | | | | | | |
| Carbohydrate | Lactose | Lactose | Lactose | Lactose | Lactose | Lactose/ |
| type | | | | | | maltodextrin |
| Carbohydrate | 9.85 | 10.7 | 10.7 | 11.6 | 11.6 | 10.6 |
| content | | | | | | |
| (g/100 kcal) | | | | | | |
| Prebiotic | GOS/FOS | GOS/FOS | GOS/FOS | GOS/FOS | GOS/FOS | GOS/FOS |
| Lipid type | Milk/veg | Milk/veg | Milk/veg | Milk/veg | Milk/veg | Veg |
| Lipid content | 5.6 | 5.4 | 5.4 | 5.1 | 5.1 | 5.6 |
| (g/100 kcal | | | | | | |
| LC-PUFA | DHA/ARA | DHA/ARA | DHA/ARA | DHA/ARA | DHA/ARA | DHA/ARA |
| Probiotic type | None | Optional | B. lactis | B. lactis | B. lactis | B. lactis |
| | | B. Lactis | | | | |
| Probiotic content | | Optional | 10e4 | 2.10e7 | 2.10e7 | 2.10e7 |
| (cfu/g) | | 10e4 | | | | |

### Example 5

The below table provides an example of the array of compositions of the invention with 4 compositions (first, second, third and fourth compositions). A similar array according to the invention is exemplified by the first, second and third compositions of the below table without the fourth composition.

| | 1 | 2 | 3 - 6 | 7-12 |
|---|---|---|---|---|
| Nutrition compositions | month | month | months | months |
| Energy (kcal/100 mL) | 67 | 65 | 63 | 63 |
| Protein (g/100 mL) | 1.51 | 1.31 | 1.18 | 1.17 |
| Protein (g/100 kcal) | 2.25 | 2.00 | 1.88 | 1.85 |
| Whey:Casein ratio | 70:30 | 70:30 | 70:30 | 50:50 |
| Lipids (g/100 mL)² | 3.78 | 3.37 | 3.22 | 3.53 |
| Carbohydrates (g/100 | 6.74 | 7.35 | 7.32 | 6.62 |
| mL) | | | | |
| Lactose (%) | 100 | 100 | 100 | 100 |
| Minerals | | | | |
| Sodium (mg/100 mL) | 30.22 | 27.60 | 24.48 | 23.65 |
| Calcium (mg/100 mL) | 52.95 | 46.42 | 44.06 | 46.12 |
| Iron (mg/100 mL) | 0.72 | 0.68 | 0.64 | 1.02 |
| Zinc (mg/100 mL) | 0.76 | 0.70 | 0.67 | 0.57 |
| Vitamins | | | | |
| A (μg/100 mL) | 69.27 | 67.75 | 62.42 | 69.78 |
| D (μg/100 mL) | 0.95 | 0.94 | 0.90 | 0.89 |
| E (μg/100 mL) | 1.26 | 1.20 | 1.28 | 1.47 |
| B-6 (mg/100 mL) | 0.03 | 0.03 | 0.03 | 0.04 |
| B-12 (μg/100 mL) | 0.18 | 0.17 | 0.18 | 0.15 |
| Folic acid (μg/100 mL) | 11.96 | 11.92 | 9.80 | 10.88 |

¹All values are means or percentage (%)
²The source of lipids was a mixture of milk fat, sunflower oil, rapeseed oil, high-oleic sunflower oil, coconut oil, fish oil (source of docosahexaenoic acid) and fungal oil (source of arachidonic acid).

It is to be noted that the various compositions of the examples can be combined together to from other arrays of composition or kits of compositions according to the invention, as long as they fulfil the below independent claims defining the invention.

All the examples above refer to nutritional compositions in a dry powder format (and after dilution as recommended as for the/ml values). The same examples can be executed in liquid form, ready-to-consume.

Having thus described the present invention in detail and the advantages thereof, it is to be understood that the detailed description is not intended to limit the scope of the invention thereof.

1. An array of nutritional compositions for use, by infants/children in their first 2 years of life, in the prevention of sub-optimal or unhealthy body composition, the array comprising:
a first infant composition for use during the first month of life,
a second infant composition for use during the 2^{nd} month of life,
a third infant composition for use during the third to sixth months of life, and
wherein the protein content (in g protein/100 kcal) of the first composition is higher than the protein content of the second composition, and
wherein the protein content (in g protein/100 kcal) of the second composition is higher or equal to the protein content of the third composition.

2. The array of compositions of claim 1:
wherein the fat content (in g fat/100 kcal) of the first composition is higher than the fat content (in g fat/100 kcal) of the second composition; and
wherein the fat content (in g fat/100 kcal) of the second composition is higher than the fat content (in g fat/100 kcal) of the third composition.

3. The array of compositions of claim 1:
wherein the energy density (in kcal/100 ml once reconstituted) of the first composition is higher than the energy density of the second composition; and
wherein the energy density (in kcal/100 ml once reconstituted) of said second composition is higher than the energy density of said third composition.

4. The array of nutritional compositions of claim 1 wherein the prevention of sub-optimal or unhealthy body composition comprises modulating the fat mass and/or the fat-free mass of said infants/children.

5. The array of nutritional compositions of claim 1 wherein said modulation induces a fat-mass and/or fat-free mass of the infants/children that is not statistically different from the fat-mass, respectively fat-free mass, of infants of similar genetic/ethnic origins that have been exclusively breast-fed during the first 4 or first 6 months of life.

6. The array of nutritional compositions of claim 1 wherein the prevention of sub-optimal or unhealthy body composition is measurable at an age of between 2 and 12 months.

7. The array of nutritional compositions of claim 1 wherein the prevention of sub-optimal or unhealthy body composition induces later in life effect.

8. The array of nutritional compositions of claim 1 wherein the prevention of sub-optimal or unhealthy body composition is accompanied by C-peptide, grelin, IGF-1, insulin, and/or leptin levels that are equal or similar to the levels exhibited by infants that have been exclusively breast-fed during the first 4 months of life.

9. The array of nutritional compositions of claim 1, wherein any of the first, second, or third compositions comprises long-chain polyunsaturated fatty acids selected from the group consisting of docosahexaenoic acid (DHA), arachidonic acid (ARA), oligosaccharides, probiotics, prebiotics and mixtures thereof

10. A method for providing nutrition to an infant in at least the first year or the first two or first 3 years of life comprising feeding to an infant an array of nutritional compositions comprising a first infant composition for use during the first month of life, a second infant composition for use during the 2^{nd} month of life, a third infant composition for use during the third to sixth months of life, and wherein the protein content (in g protein/100 kcal) of the first composition is higher than the protein content of the second composition, and wherein the protein content (in g protein/100 kcal) of the second composition is higher or equal to the protein content of the third composition at the corresponding ages.

11. The method according to claim 10, wherein the nutritional compositions are packed in single dose units, each single dose unit comprising sufficient nutritional composition to prepare a single serving upon reconstitution with water.

12. An age-tailored nutrition kit for infants and young children comprising the array of nutritional compositions according to claim 1, wherein the nutritional compositions are packed in single dose units.

13. The kit of claim 12, wherein the nutritional compositions are packed together in a single packaging.
